(19) United States
(12) Patent Application Publication
(10) Pub. No.: US 2009/0123523 A1
(43) Pub. Date: May 14, 2009

(54) PHARMACEUTICAL DELIVERY SYSTEM

(75) Inventor: Gail C. Kopp, Mahwah, NJ (US)

Correspondence Address: HOXIE & ASSOCIATES LLC, 75 MAIN STREET , SUITE 301, MILLBURN, NJ, 07041, US

(73) Assignee: G. Coopersmith LLC, Mahwah, NJ (US)

(21) Appl. No.: 12/270,022

(22) Filed: Nov. 13, 2008

Related U.S. Application Data
(60) Provisional application No. 60/987,638, filed on Nov. 13, 2007.

(51) Int. Cl. A61K 9/00

(52) U.S. Cl. 424/439; 424/442

(57) ABSTRACT

The present invention is directed to methods and compositions for administering medicaments to large animals by enveloping a medicament within a food stuff.

### CROSS REFERENCE TO RELATED APPLICATIONS

This application claims priority to U.S. Provisional Application No. 60/987,638 filed Nov. 13, 2007, the contents of which are incorporated herein by reference.

### FIELD OF THE INVENTION

The present invention relates to a pharmaceutical delivery system for the administration of medicaments to large animals.

### BACKGROUND OF THE INVENTION

Animals frequently refuse to ingest the medicaments due to the medication's unusual or repugnant odor, taste, and/or texture. The primary way of administering veterinary medications to reluctant animals orally is by concealing the medication in food; however, there are various shortcomings with this method. The process is time consuming and messy, as foods must be prepared, e.g., cut and cored, and mixed with other agents, e.g., molasses or sugar solutions. Furthermore, there is a significant risk that the medication will fall out of the food, either by improper concealment, or by the animal manipulating the food to expel the medicament. Additionally, animals having keen taste and olfactory senses, e.g., equines, are often able to discern if a medicament is concealed in a food product, resulting in the animal refusing to ingest the food or medicament. Also, concealing medicaments in food only works for solid dosage forms, as there is no convenient way to conceal liquid medicaments in a food. Thus, liquid dosage forms are typically administered via an oral syringe.

If concealment of medicaments in food fails, medicaments are administered by methods which are unpleasant for both the animal and the animal handlers. Such methods include directly injecting the medicament into the stomach cavity by e.g., nasal-gastric tube. Another method is to process the medicament into a paste, e.g., tablets or pills are crushed and mixed with a carrier, and the paste is administered via syringe into the oral cavity. The paste generally has a viscosity such that it will stick to the tongue and gums, and eventually be swallowed by the animal; however, these procedures are even more time consuming than embedding medicaments in food. When force feeding an animal, e.g., by syringe, the animal may need to be physically restrained, as medicament is forced down the animal's throat. This is a daunting task, especially for large animals, such as a horse. More than one handler may be required to restrain the animal, as other handlers attempt to force multiple pills, sometimes several times a day, into the animals throat. Additionally, there exists a real possibility for animal handlers to be injured in the process.

Administration of medicaments via a syringe has additional shortcomings. Some medicaments cannot be crushed, as tablets with special coatings, or special time-release tablets must be given whole. Additionally, during the crushing steps, there is a danger that the medicament will be inhaled by the handler during processing of the medicament, as many these medicaments are toxic to humans. In shy or nervous horses, the insertion of the syringe into the mouth can cause the horse to lift its head above the handler's arm reach. Even if a horse initially accepts the paste, it may be spit right back out, especially if the handler does not clear the horse's mouth of chewed food matter. If the animal does not consume all of the paste, the desired blood levels of the medicament are not achieved, thereby reducing or eliminating any benefit the medicament is administered for. Another problem is that medicaments should be completely consumed at once. If the medicament consumed over a long period of time, the timing of administration is skewed, and desired blood levels of the medication are not reached. Thus, it is still preferred to conceal medicaments within a food, and then feed the food stuff to the animal to avoid such dangerous procedures.

Although some systems for concealing medicaments in food have been developed for delivery of drugs to smaller animals, a unique problem exists with administering medicaments to larger animals. A large animal may have a larger oral cavity, so that the animal will be able to separate the medicament from the food product, and reject the medicament while consuming the food product. This problem is compounded by the fact that medicaments, e.g., tablets, for larger animals are quite large, as they should be because the animal too is quite large. Thus, when concealing and administering horse sized medicaments to a horse, the horse has the ability to work the medicament back out, usually spitting it onto the floor, or the handler.

Accordingly, there needs to be methods to solve these and other problems with administering embedded medicaments to large animals. There is a need to develop improved methods of administering medicaments to animals by hiding medicaments in food so as to avoid resorting to barbaric and potentially dangerous methods of administration should concealment fail.

### SUMMARY OF THE INVENTION

The present invention is directed to an oral delivery system that allows for the administration of medications to large animals, wherein the delivery system ensures that the total dose of medication is delivered, e.g., consumed. The present invention conceals a medicament in a food stuff which masks the odor, taste, and/or texture of the medicament, which aids in the administration of the medicament especially if the animal is particularly sensitive to the taste or odor of the medicament. The present invention provides for methods to prevent the animal from separating and rejecting the medicament from the food stuff, even if the animal has a large oral cavity and if the medicament is large. The present invention provides a safer and more effective way to orally administer medicaments to a large animal. The present invention also provides a method to administer medicaments in various dosage forms, e.g., tablets, capsules, powders, liquids, etc.

One embodiment of the present invention is directed to Method 1.0: A method for administering a medicament to an animal comprising: applying a medicament on an adhesive surface of a flexible food stuff; folding over said flexible food stuff to form a pouch having at least two walls each having an inner surface formed by said adhesive surface such that said medicament is enveloped within said pouch; and administering said pouch containing said medicament orally to an animal for ingestion.

Additional embodiments of the present invention include Methods:
1.1 Of method 1.0, further comprising applying compression force to said inner surfaces of said pouch to cause said adhesive surface to engage said medicament prior to administering said pouch to said animal.
1.2 Of any of methods 1.0 of 1.1 wherein said food stuff has a midline along the adhesive surface, and the food stuff is folded along the midline to form the pouch.
1.3 Of any of method 1.2 wherein the medicament is placed on the midline.
1.4 Of any of methods 1.0-1.3 wherein the adhesive surface is formed by applying an adhesive on to a surface of the food stuff.
1.5 Of method 1.4 wherein the medicament is engaged with the adhesive.
1.6 Of methods 1.4 or 1.5 wherein the medicament is applied on top of the adhesive.
1.7 Of any of methods 1.0-1.6 wherein the medicament is in a solid form or paste.
1.8 Of any of methods 1.0-1.6 wherein the flexible food stuff further comprises a bottom surface opposite said adhesive surface, the bottom surface being substantially impermeable to liquids and forming an outer surface of said at least two walls when said food stuff is folded as a pouch.
1.9. Of any one of methods 1.0-1.6 or 1.7 wherein said medicament is in liquid form, and wherein said food stuff absorbs said liquid.
1.10. Of any of methods 1.0-1.9 wherein said adhesive surface is substantially flat.
1.11 Of any of methods 1.4-1.10 wherein said pouch has a plurality of edges and the application of force to said inner surfaces causes said adhesive to herniated beyond at least one edge.
1.12. Of any of methods 1.0-1.11 wherein the food stuff is substantially planar in shape.
1.13. Of any of methods 1.0-1.12 wherein the adhesive surface comprises an adhesive area circumscribing a non-adhesive area.
1.14 Of any of methods 1.0-1.13 wherein the flexible food stuff has a bottom surface opposite said adhesive surface, wherein the bottom surface is coated with a flavorant.
1.15 Of any of methods 1.14 wherein the flavorant is a sugar.
1.16 Of any of methods 1.0-1.15 wherein the medicament is admixed with an adhesive to form a paste, and the paste is applied to the food stuff.
1.17 Of any of methods 1.0-1.16 wherein the food stuff is rolled into a substantially cylindrical shape.

Another embodiment of the present invention is directed to Method 2.0: a method for administering a medicament to an animal comprising applying an adhesive on the top of a first food stuff to form an adhesive surface; engaging a medicament with said adhesive surface; engaging the top surface of a second food stuff on top of said adhesive surface and medicament; and applying compressive pressure to the first and second food stuff to engage said food stuff together and envelope said medication within said food stuff.

Additional embodiments of the present invention include Methods:
2.1 Of method 2.0 further comprising applying compressive pressure to the bottom surface of the first and second flexible food stuff to engage first and second food stuffs together and envelope said medication within the first and second food stuffs.
2.2. Of method 2.0 or 2.1 wherein the top surfaces are substantially flat.
2.3 Of any of methods 2.0-2.2 wherein the first and second food stuff further comprises an edge, and application of compressive force to the bottom surfaces causes the adhesive become herniated beyond said edge.
2.4 Of any of methods 2.0-2.3 wherein said first or second food stuff is substantially planar in shape.
2.5 Of any of methods 2.0-2.4 wherein said top surfaces of said first and second food stuff are substantially complementary in size and shape to one another.

Other embodiments of the present invention include Kit 3.0: a kit comprising a flexible food stuff having a top surfaces coated with a layer of adhesive, said adhesive layer reversibly bound to a film layer; and instructions for the preparation and use of said flexible food stuff for the administration of a medicament to an animal comprising removing said film layer from said flexible food stuff, engaging a medicament on to said food stuff, folding said flexible food stuff to envelope said medicament within said adhesive and food stuff to form a pouch, and feeding the pouch to an animal.

Other kits of the present invention include kits:
3.1 Of kit 3.0 further comprising a second flexible food stuff having a top surface coated with a layer of adhesive, the film layer having first and second sides, the second side of the film layer each being reversibly bound to the adhesive layer of the second food stuff.
3.2. Of kit 3.0 or 3.1 wherein a majority of said adhesive remains bound to said food stuff following removal said film from said adhesive layer.

In another embodiment of the present invention, a kit is provided comprising: a flexible food stuff having an adhesive surface; and instructions for the preparation and use of said flexible food stuff for the administration of a medicament to an animal comprising engaging a medicament on to said adhesive surface, folding said flexible food stuff to envelope said medicament within said food stuff to form a pouch, and feeding the pouch to an animal. The kit may further comprising an edible adhesive separately packaged from the food stuff, and instructions on applying said adhesive on to said food stuff to form the adhesive surface.

The kits of the present invention may also comprise one or more medicaments.

The present invention may be a pharmaceutical delivery system comprising a food stuff having an adhesive surface engaging a medicament, said food stuff in the shape of a pouch having two walls such that said medicament is enveloped within said pouch and said two walls are engaged with one another. The pouch may be formed with a single piece of flexible food stuff, or by a first and second food stuff substantially complementary in size and shape to one another. Generally, the walls of the pouch are engaged by the adhesive surface. The food stuff may comprise a partially caramelized sugar, and/or licorice extract.

The adhesive used in the methods, kits and systems of the present invention may be selected from the group consisting of honey, molasses, maple syrup, corn syrup, cane juice, rice syrup, licorice extract, peanut butter, mashed or pulverized fruits (e.g., banana, cherry, strawberry, apple, etc.), caramel, sugar, oil, dairy products (e.g., condensed milk, yogurt, cheese), or mixtures thereof.

The medicament in the present invention may be in a delivery form selected from the group consisting of tablets, capsules, powders, pastes, liquids, or mixtures thereof. In one embodiment, the medicament may be admixed with an adhesive to form a paste, and the paste is deposited on to the flexible food stuff. For example, if the medicament is in the form of a tablet, the tablet is crushed and admixed with an adhesive to form a paste, and the paste is then deposited on to the food stuff. For example, if the medicament is in the form of a capsule, the capsule is emptied and the contents are admixed with an adhesive to form a paste, and the paste is then deposited on to the food stuff. For example, if the medicament is in the form of a paste, the paste may be admixed with an adhesive, and deposited on to the food stuff. The adhesive used to form the paste may be the same or different adhesive on the food stuff. The amounts of adhesive required to produce the paste will depend on the adhesive and medicament, and can be easily determined by one of skill in the art.

The present invention also includes the use of any kit or delivery system described herein in administering one or more medicaments to an animal. The animal in the present invention may be any animal, e.g., a mammal, e.g., an equine.

The present invention may be used when the handler would prefer a non-force medication administration method, when the handler is alone when medicating the animal, when the animal rejects other methods of medicament administration, when the animals presents a danger to the handler, when the treatment regime requires confirmation that the medicament was ingested timely and completely, when the animal is on a long-term treatment plan and requires some variety in the delivery method, and/or when the handler desires to make the animal's life more pleasant with an easy medication method.

Other embodiments of the present invention will be apparent to one of skill in the art.

### DETAILED DESCRIPTION OF THE INVENTION

The present invention is directed in part to an oral medicament delivery system for an animal comprising a medicament engaged with and enveloped by a food stuff. The medicament is engaged within the food stuff to prevent the animal from separating and rejecting the medicament while consuming the food stuff. The medicament is enveloped within the food stuff to mask any odor, taste or texture which may be perceived by the animal when the medicament is presented for administration.

In one embodiment, the food stuff comprises a top adhesive surface to receive and fix the medicament. The food stuff is then folded to allow the adhesive top surface to adhere to itself, effectively engaging and enveloping the medicament within the adhesive surface and food stuff. Compressive force may then be applied to the adhesive surfaces to seal the layers of food stuff together to prevent the animal from separating the layers of the food stuff, and further, from separating the medicament, e.g., from the food stuff.

In one embodiment, an adhesive is applied to a surface of the food stuff to form the adhesive surface to receive and fix the medicament. The food stuff is folded to allow the adhesive surface to adhere to itself, effectively engaging and enveloping the medicament within the adhesive and the food stuff. Application of compressive force to the adhesive surface may cause excess adhesive to become herniated beyond one or more edges of the food stuff. The excess adhesive may also be used as a to cause the food stuff (containing the medicament) to adhere to the mouth of the animal following administration, ensuring that the whole food stuff, and hence the total dose of medication, is consumed.

In another embodiment, two separate food stuffs are provided wherein at least one food stuff has an adhesive surface. A medicament is engaged on the adhesive surface, e.g., in the middle of the adhesive surface, and the second food stuff is laid on top of the medicament and adhesive surface so that the medicament is engaged with the adhesive surface and enveloped between the two food stuffs. Application of compressive force to the two separate food stuffs will cause the food stuff to become engaged with one another.

In another embodiment, an adhesive may be applied to at least one food stuff to form the adhesive surface. In another embodiment, two separate food stuffs are provided, each having an adhesive surface. In still another embodiment, an adhesive may be applied to two separate food stuffs to form an adhesive surface on each food stuff. Application of compressive force to the two separate food stuffs may cause excessive adhesive to become herniated beyond one or more edges of the food stuff, which may assist in administering the food stuff (containing the medicament) to an animal.

The present invention may be used for any animal, preferably an animal whose diet is be controlled by humans. These animals may include, e.g., domesticated farm animals (e.g. bovine, equine, porcine, etc.) as well as undomesticated animals held in captivity, e.g. in zoological parks and the like. Preferably, the animal is a large animal, e.g., bovine or equine.

“Engaged,” “engagement,” and like terms refers to the fixing of one material or surface to another material or surface in a substantially relative position. Thus, for example, a medicament is engaged with a food stuff if position of the medicament relative to the food stuff is constant. It is contemplated that a liquid medicament may be engaged by an adhesive surface, an adhesive, or food stuff when the liquid medicament is absorbed by the adhesive surface, adhesive, or food stuff, such that the liquid medicament does not leach out of the adhesive surface, adhesive, or food stuff.

“Enveloped”, “enveloping” and like terms refers to one component being substantially or completely surrounded by another component. Thus for example, a medicament enveloped within an adhesive is substantially or completely enclosed in the adhesive.

A “food stuff” is a food composition which includes ingredients which are suitable for ingestion and/or digestion by an animal. The preferred material used to form the food stuff may be any material known by those of skill in the art. The food stuff may comprise protein (e.g., meats, meat by-products, dairy products), fat, carbohydrates, soybean, corn, wheat, etc. Preferably, the food stuff is flexible and pliable, and frangible when chewed by an animal. Preferably, the food stuff used in present invention has a pleasant odor and taste or may include odor and/or taste masking agents to render the medicaments to be contained therein more palatable. Preferably the food stuff promotes salivation, so that the entire food stuff, and hence the medicament, will be consumed.

In some embodiments, the moisture content of the food stuff is from about 1% to about 40% of the total weight of the composition, e.g., from about 5% to about 30%, from about 5% to about 20%, from about 5% to about 15%, and about 15%. Preferably the moisture content is less than about 10%.

The protein in the food stuff may be supplied by any of a variety of sources, including plant sources, animal sources, or both. Animal sources include, for example, meat, meat by-products, seafood, dairy, eggs, etc. Meats include, for example, the flesh of poultry; fish; and mammals (e.g., cattle, pigs, sheep, goats, and the like). Meat by-products include, for example, lungs, kidneys, brain, livers, and stomachs and intestines (freed of all or essentially all their contents). Preferably, animal source proteins are not administered to herbivores, and proteins sourced from an animal species are not fed to the same species of animal. The protein content of the food stuff may be from about 1% to about 80%, e.g., from about 1% to about 50%, from about 2% to about 40%, from about 3% to about 20%. Preferably, the protein content of the food stuff is greater than about 4.7%, e.g., from about 4.7% to about 10%.

Fat, if present in the food stuff, can be supplied by any of a variety of sources, including meat, meat by-products, fish oil, and plants. Plant fat sources include wheat, flaxseed, rye, barley, rice, sorghum, corn, oats, millet, wheat germ, corn germ, soybeans, peanuts, and cottonseed, as well as oils derived from these and other plant fat sources. The fat content of the food stuff may be from about 1% to about 40%. Preferably, the fat content of the food stuff is greater than about 2.3%, e.g., from about 2.3% to about 5%.

Carbohydrate, if present in the food stuff, may be supplied by any of a variety of sources, including oat fiber, cellulose, peanut hulls, beet pulp, parboiled rice, corn starch, corn gluten meal, and any combination of those sources. Grains supplying carbohydrate include, but are not limited to, wheat, corn, barley, and rice. The carbohydrate percentage may be calculated as nitrogen free extract (“NFE”), which may be calculated as follows: NFE=100%−moisture %−protein %−fat %−ash %−crude fiber %. The carbohydrate content of the food stuff may be greater than about 50%, e.g., from about 50% to about 90%, from about 60% to about 85%, or from about 75% to about 85%. Preferably, the carbohydrate content of the food stuff is about 80%, or about 81%.

“Ash” consists of compounds that are not organic or water, generally produced by combustion of biological materials. Ash may be determined by any number of methods known by those of skill in the art. The ash content of the food stuff is may be from about 1% to about 10%, e.g., 2% to about 7%, about 3%, or about 4%. Preferably the ash content of the food stuff is less than about 3%.

The food stuff may also contain nutraceuticals, nutrigenomic agents, vitamins and minerals to supplement the animal's diet in order to avoid deficiency and maintain health. Specific vitamins and minerals, and amounts for to avoid deficiency known by those of skill in the art, for example, as published by The Association of American Feed Control Officials (AAFCO). Vitamins useful include, e.g., vitamin A, vitamin B1, vitamin B2, vitamin B6, vitamin B12, vitamin C, vitamin D, vitamin E, vitamin H (biotin), vitamin K, folic acid, inositol, niacin, and pantothenic acid. Minerals and trace elements useful as food additives include calcium, phosphorus, sodium, potassium, magnesium, copper, zinc, choline, and iron salts.

The food stuff may further contain additives known in the art. Such additives should be present in amounts that do not impair the purpose and effect provided by the invention. Examples of additives include substances with a stabilizing effect, organoleptic substances, processing aids, and substances that provide nutritional benefits. Stabilizing substances may increase the shelf life of the composition. Suitable examples can include preservatives, antioxidants, synergists and sequestrants, packaging gases, stabilizers, emulsifiers, thickeners, gelling agents, and humectants. Examples of emulsifiers and/or thickening agents include gelatin, cellulose ethers, starch, starch esters, starch ethers, and modified starches. A humectant may be incorporated into the food stuff to enhance the flexible texture and retain moisture when the composition is stored. Humectants are known in the art, and may include, e.g., glycerine and sorbitol.

Additives for coloring, palatability, and nutritional purposes can include colorants, salts (including but not limited to sodium chloride, potassium citrate, potassium chloride, and other edible salts), vitamins, minerals, and flavoring. The amount of such additives in a composition typically is up to about 5% by weight (on a dry matter basis of the composition). Other additives can include nutraceuticals, antioxidants, omega-3 fatty acids, omega-6 fatty acids, glucosamine, chondroitin sulfate, vegetable extracts, herbal extracts, and oral care additives such as anti-plaque compounds, anti-tarter compounds, and anti-gingivitis compounds, etc.

In one embodiment, the flexible food stuff has an adhesive surface, and a bottom which is opposite to the adhesive surface. The bottom may be coated with a flavorant to increase the palatability of the food stuff. The flavorant may be any flavor or material commonly fed to the animal, such as fruit extracts, and sugar. A preferred flavorant may be a sugar, such as glucose, maltose, sucrose, lactose, fructose, or a mixture thereof.

Methods of manufacturing the food stuff is well known in the art, and may be accomplished in any number of methods. In one embodiment, dry ingredients, including, for example, animal protein sources, plant protein sources, grains, etc., are ground and mixed together. Moist or liquid ingredients, including fats, oils, animal protein sources, water, etc., are then added to and mixed with the dry mix. The mixture is then processed into an appropriate shape by methods known by those of skill in the art, e.g., by extrusion or rolling.

The food stuff may be of any shape. Preferably, at least one surface is substantially flat, e.g., a top surface, and the top surface has adhesive properties. Preferably, the food stuff is substantially planar in shape, e.g., a flat sheet.

The food stuff may have two halves of substantially similar size and shape so that the may be food stuff folded in half. Alternatively, the food stuff may be folded multiples times, e.g., the food stuff has three or more panels so that it may be folded a plurality of times so that the medicament is buried within the interior of the structure. Alternatively, the medicament is placed on the adhesive surface of the food stuff, and the food stuff is “rolled” into a cylindrical shape.

When the present invention utilizes the joining of two food stuffs, each food stuff may be of any shape. For example, the food stuff may be shaped as any polyhedron, and one surface of one polyhedron is adhesive for placement of the medicament. The adhesive surface is then matched with another polyhedron, and the two separate portions are joined together to form a food stuff enveloping a medicament in the shape of, e.g., a polyhedron, e.g., a cube or cuboid. The food stuff may be two hemispheres of a sphere, wherein the top surface of hemisphere of one sphere is adhesive, and the two hemisphere and joined together to form a sphere, wherein the medicament is engaged and enveloped in the food stuff. Preferably the two food stuffs are of similar shape and size so that the surface having the adhesive joins the entire surface.

In one embodiment, a food stuff is a flat sheet, e.g., substantially planar, having a bottom surface, an adhesive top surface, and a mid-line on the top surface. A medicament is placed and engaged on the mid-line, and the sheet is folded in half along the mid-line to completely envelope the substance between two walls to form a pouch. Thus, the adhesive top surface becomes the inner walls of the pouch, and the bottom surface becomes the outer walls of the pouch. The walls are then sealed together, preferably by applying pressure to the outer walls to, e.g., further engage and envelope the medicament in the adhesive, and allow the opposing wall to engage each other to prevent separation of the walls and removal of the medicament from the interior.

The adhesive surface in the present invention may be created by any method known by those of skill in the art. In one embodiment, the food stuff has adhesive properties, e.g., the food stuff may self-adhere to itself and a medicament. In another embodiment, an adhesive may be applied to a surface to impart adhesive properties to the surface. Adhesives suitable for use in the present invention include those which are non-toxic to the animal following ingesting. The adhesive should be of sufficient strength to engage the food stuff and medicament, and may be in a solid, liquid, or viscous liquid form.

The food stuff of the present invention may be provided as a kit containing a food stuff, one or more adhesives to be applied to the food stuff, and instructions for use. The adhesive may be packaged separately, e.g., in a tube or jar separate from the food stuff.

The food stuff of the present invention may be provided as kit containing a food stuff with an adhesive surface, a film layer in substantial continuous contact with the adhesive surface, and instructions for use. The film layer may be removed from the adhesive surface, and the surface has sufficient adhesive properties to engage the medicament. One or more medicaments are placed on the adhesive surface, and the food stuff is folded to envelope the medicament as described herein. The film layer may be any film known in the art, e.g., wax or parchment paper.

In one embodiment of the present invention, the food stuff may be supplied in strips comprising more than one sheet of food stuff, wherein the strip comprises a series of perforations separating each sheet. When desired, an individual sheet of food stuff may be removed along the perforations. A food stuff having an adhesive surface may also be supplied in a strip with a film layer in substantial continuous contact with the adhesive surface.

In one embodiment, the food stuff is extruded into sheets of suitable size depending on the size of the animal. Thus, for example, food stuff suitable for a horse may be extruded in 4″×4″×⅛″ sheets. The food stuff may be provided with an adhesive surface or an adhesive to be applied on to the surface.

The present invention is particularly advantageous in aiding in the administration of a medicament comprising at least one pharmacologically active agent formulated in a pharmaceutical dosage form suitable for oral administration. Suitable pharmaceutical oral dosage forms include, but are not limited to, the conventional forms such as tablets, capsules, powders, liquids, pastes, and the like. In addition, the medicament could be of a non-medicinal type, such as a particular food stuff, e.g., fruits, vegetables. The term medicament is meant to encompass, e.g., acetylsalicylic acid, acetaminophen, vitamins, nutraceuticals, and medications which are psychotropic, anti-hypertensitive, anti-seizure, amphetamine, anti-microbial, anthelminitic, antibiotic, anti-viral, anti-retroviral, anti-fungal, anti-depressant, stimulants, anti-histamine, anti-anxiety, tricyclics, tranquilizers, benzodiazepines, hypnotics, mood stabilizers, codeine, selective serotonin reuptake inhibitors, anti-allergy, phenothiazine, chemotherapeutics, amines, monoamine oxidase inhibitors, anti-carcinogens, analgesics, muscle relaxants, ergot preparations, anti-cholinergic, anti-inflammatory, anti-gout preparations, soporfic, hormonal preparations, appetite suppressants, analgesics, muscle relaxants, opioids, nutraceuticals, and nutrigenomic compounds. It may be appreciated that it may be prohibited to administer certain medicaments to certain animals, e.g., depending on the purpose of the animal. For example, administering steroids and opioid to horses used for racing is generally prohibited; however, the present invention contemplates that such medicaments may be used in combination with the present invention, as the present invention is not limited to specific animal species, or purposes of the animals.

The present invention is suitable for the administration of medicaments in various forms, such as tablets, capsules, and liquids. Solid dosages, e.g., tablets, capsules, and powders need only be sprinkled on the adhesive surface of the food stuff. Liquid dosages, including gels and pastes, need only be applied on the adhesive surface and allowed to be absorbed into, e.g., the adhesive or the food stuff. Some liquid dosage forms, e.g., a gel, may not be absorbed into the food stuff, and thus the food stuff is folded as if the liquid dosage were a solid dose format. The medicament is preferably crushed and sprinkled on to the adhesive surface if the medicament is in a tablet form; however, the handler should confirm that the tablet may be crushed.

In one embodiment, the present invention allows for the administration of one or more than one medicament to the animal simultaneously. One of skill in the art need only engage one or more medicaments on an adhesive surface of the food stuff, and then process the food stuff as disclosed herein.

It is contemplated herein that the present invention is not limited to the administration of medicaments. For example, a treat, e.g., fruit, may also be engaged and enveloped in the food stuff, either alone, or in combination with a medicament or other treats.

Preferably, the mass of the food stuff is greater than the mass of the medicament being administered so that most of the mass being consumed is the food stuff, e.g., more than twice the mass of food stuff compared to medicament is consumed at once. Thus, for example, the food stuff may be more than about 1.0× the mass of the medicament being administered, e.g., about 1.5×, about 2×, about 3×, about 4×, about 5×, about 10×, 15×, about 20×, about 25×, about 50×, or about 100× the mass of the medicament.

### EXAMPLE 1

A food stuff is prepared containing molasses, wheat flour, brown rice syrup, cane juice, sugar, non-hydrogenated palm oil, salt, licorice extract, caramel color and flavors. The food stuff is extruded and baked into ⅛″ thick sheets. The sheets are then cut into 4″×4″ squares. The weight of each square is approximately 68 grams having the composition as presented in Table 1.

TABLE 1

| | PER 100 |
|---|---|
| | grams of food |
| NUTRIENT | stuff |
| Calories (kcal) | 364.00 |
| Calories from Fat (kcal) | 20.43 |
| Calories from saturated Fat (kcal) | 10.26 |
| Protein (g) | 4.68 |
| Carbohydrate (g) | 81.30 |
| Dietary fiber (g) | 1.99 |
| Fat (g) | 2.27 |
| Saturated Fat (g) | 1.14 |
| Water (g) | 8.81 |
| Calcium (mg) | 348.00 |
| Iron (mcg) | 5.47 |
| Sodium (mg) | 120.00 |
| Fructose (g) | 3.99 |
| Glucose (g) | 6.23 |
| Maltose (g) | 6.93 |
| Sucrose (g) | 22.35 |
| Ash (g) | 2.97 |

The matters set here are offered by way of illustration only and not as limitations. While particular embodiments have been shown and described, it will be apparent to those skilled in the art that changes and modifications may be made without departing from the broader aspects of invention. The actual scope of the protection sought is intended to be defined in the following claims when viewed in their proper perspective based on the prior art.

1. A method for administering a medicament to an animal comprising:
engaging a medicament on an adhesive surface of a flexible food stuff;
folding over said flexible food stuff to form a pouch having at least two walls each having an inner surface formed by said adhesive surface such that said medicament is enveloped within said pouch and said at least two walls are engaged with one another;
administering said pouch containing said medicament orally to an animal.

2. The method of claim 1 wherein said food stuff further comprises at least two panels, wherein at least one panel has said adhesive surface.

3. The method of claim 1 further comprising directing compressive force to said inner surfaces of said pouch.

4-5. (canceled)

6. The method of claim 1 wherein said adhesive surface is formed by applying an adhesive on to a surface of said food stuff.

7.-8. (canceled)

9. The method of claim 1 wherein said adhesive surface is substantially flat.

10-12. (canceled)

13. The method of claim 1 wherein said adhesive surface comprises an adhesive area circumscribing a non-adhesive area.

14. A method for administering a medicament to an animal comprising:
applying an adhesive on the top of a first food stuff to form an adhesive surface;
engaging a medicament with said adhesive surface;
engaging the top surface of a second food stuff on of said adhesive surface and medicament;
applying compressive pressure to the first and second food stuff to engage said food stuff together and envelope said medication within said first and second food stuffs.

15-17. (canceled)

18. The method of claim 1 wherein the adhesive is selected from the group consisting honey, molasses, maple syrup, corn syrup, cane juice, rice syrup, licorice extract, peanut butter, mashed or pulverized fruits, caramel, sugar, oil, dairy products, or mixtures thereof.

19. (canceled)

20. The method of claim 1 wherein said animal is an equine.

21. The method of claim 1 wherein said medicament is in a delivery form selected from the group consisting of tablets, capsules, powders, liquids, pastes, or mixtures thereof.

22. The method of claim 1 further comprising applying a second medicament to the adhesive surface.

23. (canceled)

24. A kit comprising:
a flexible food stuff having a top surfaces coated with a layer of adhesive, said adhesive layer reversibly bound to a film layer; and
instructions for the preparation and use of said flexible food stuff for the administration of a medicament to an animal comprising removing said film layer from said flexible food stuff, engaging a medicament on to said food stuff, folding said flexible food stuff to envelope said medicament within said adhesive and food stuff to form a pouch, and feeding the pouch to an animal.

25. The kit of claim 24 further comprising a second flexible food stuff having top surface coated with a layer of adhesive, said film layer having first and second sides, said second side of said film layer each being reversibly bound to the adhesive layer of the second food stuff.

26-28. (canceled)

29. The kit of claim 24 wherein said animal is an equine.

30. The kit of claim 24 further comprising a medicament.

31-43. (canceled)

44. The method of claim 1 wherein the flexible food stuff has a bottom surface opposite said adhesive surface, wherein the bottom surface is coated with a flavorant.

45. The method of claim 44 wherein the flavorant is a sugar.

46. The method of claim 1 wherein the medicament is admixed with an adhesive to form a paste, and the paste is applied to the food stuff.
